(19) United States
(12) Patent Application Publication
(10) Pub. No.: US 2008/0044503 A1
(43) Pub. Date: Feb. 21, 2008

(54) A METHOD FOR PRODUCING FUNCTIONAL FOOD AIMED AT BLOOD PRESSURE REDUCTION FROM THE HYDROLYZED VEGETABLE SOLUTION

(76) Inventors: Pao-Chuan Hsieh, Nei Pu Hsiang (TW); Chao-Yu Lai, Nei Pu Hsiang (TW)

Correspondence Address: LEONG C LEI, PMB # 1008, 1867 YGNACIO VALLEY ROAD, WALNUT CREEK, CA, 94598, US

(21) Appl. No.: 11/383,497

(22) Filed: May 16, 2006

(51) Int. Cl. A61K 38/08; A61K 36/42

(52) U.S. Cl. 424/758; 514/17

(57) ABSTRACT

The present invention of “A method for producing functional food aimed at blood pressure reduction from the hydrolyzed vegetable solution, relates to study and develop methods for producing high-performance Angiotensin Convening Enzyme Inhibitor (ACEI) from vegetables by the following production, processes, such as washing, drying, powdering, ultrasonic extraction, vacuum concentration, freeze-drying, dissolution as well as enzymic hydrolysis etc., in addition, the said ACEI is acting as a kind of food additive.

### BACKGROUND OF THE INVENTION

(a) Technical Field of the Invention

The present invention relates to a method for producing Angiotensin Convening Enzyme Inhibitor (ACEI) from vegetables hydrolysate, which is acting as a food additive with a function of blood pressure reduction.

(b) Description of the Prior Art

It is known that the foodstuff contains some functional features, and also it is found in various studies that the protein taken by living organism will affect the physiology system of such living organism variously while it has been digesting by bowel enzyme, due to such protein has a specific 3-dimensional structure.

According to the recent research reports, it has pointed out that proteins filtered from some natural materials have a function of blood pressure reduction and, more over, it is expectable to reduce cholesterol in blood as well as the occurrence rate of cerebral paralysis; and the causes for hypertension are classified into congenital hypertension and acquired hypertension, in which, the primary reaction mechanism of acquired hypertension covering; angiotensin I (Ang I) generated in kidney, where it is delivered, by the blood circulation, and reacted with angiotensin enzyme (ACE) in lung, to ionize the His-Leu at C-end and hence generate angiotensin II (Ang II). According to researches, it says that the inhibiting capability of soybean protein against ACE can reach up to 90% after performing enzyme hydrolysis process by alcalase and pepsin for 1 hour, furthermore the similar circumstance could be found under the hydrolysis process with Gelatin, where there is a positive relationship between inhibiting capability against ACE and hydrolysis ratio; additionally, it is applicable to produce the peptide with a mass of about 200˜5000 kDa if the pepsin hydrolysate is processed with chromatography via a desalting column of Sephadex G-25. While filtering active peptide with HPLC, it is found that there are peptide segments, such as tryptophan, phenylalanine, and praline at C-end, and also valine, and isoleucine at N-end, which has a higher activity for ACEI.

Hypertension is a key factor in the causes of cardiovascular diseases, where among the clinic medicines for the hypertension treatment, the one has the least side-effect is Angiotensin Enzyme Inhibitor (ACEI), which also has a function of blood pressure reduction; and in light of the recent research reports, it is found that there are components existing inside some natural materials which am similar to ACEI, although it is available to extinct such components of ACEI with dissolvent individually, but in general all of there have inferior inhibition ratio, and while it is broken into small peptide, the inhibition ratio against ACE is then raising dramatically as well, however, the existing studies are focusing on performing hydrolysis process with single enzyme only and there is a minor increment in quantity of ACEI product at all.

Due to ACE (Dipeptidycardoxypeptidase, EC3.4.15.1) primarily exists in the blood vessel endothelial cells, lung, kidney and brain etc, inside human body, which is capable to convert inert angiotensin I (AngI) originally into active angiotensin II by cutting off two amino acid (His-Leu) (Hwang and Ko, 2004), and hence resulting in blood vessel constriction and blood pressure increasing, meanwhile ACE can also activate bradykinins having a function of blood vessel dilatation, and hence increasing the blood pressure consequently, thus the blood pressure increment will be relieved properly if it is applicable to block the function of this enzyme, while combining ACEI with ACE, it will then influence ACE's activity, decelerate tire generation of angiotensin II as well as the destruction of bradykinin, therefore if we can add ACEI to our regular foods then it is assure that it will have a positive effect on the treatment of hypertension disease.

### SUMMARY OF THE INVENTION

The primary purpose of the present invention lies in the method for producing a kind of food additive which is applicable to enhance capability of ACEI from vegetable extracts.

The present invention of “A method for producing functional food aimed at blood pressure reduction from the hydrolyzed vegetable solution, while performing the experiments, the optimum ACEI is resulted from the winter melon, and liquid extract of carrot.

The present invention of “A method for producing functional food aimed at blood pressure reduction from the hydrolyzed vegetable solution, wherein it is applicable to produce the peptide with a function of blood pressure reduction by a filtering process from natural vegetables and, in addition, the relevant effects can be verified upon performing animal experimentation.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic view showing the sample's extracting process in accordance with, the present invention;

FIG. 2 showing the ACEI measurement for winter melon extract, after adding a specific enzyme;

FIG. 3 is a standardized mapping for the Hippuric acid (HA);

FIG. 4 is a HPLC mapping showing a compete reaction between the substrate of Hippuric acid (HA) and ACE;

FIG. 5 is a HPLC mapping showing the circumstance while adding the vegetable crude extract liquid A (carrot);

FIG. 6 is a HPLC mapping showing the circumstance while adding the vegetable crude extract liquid B (winter melon) and

FIG. 7 is a schematic view showing the observation in variability of diastolic blood pressure (DBP) for a rat suffered from hypertension disease, while feeding it with winter melon hydrolysate for 4 weeks.

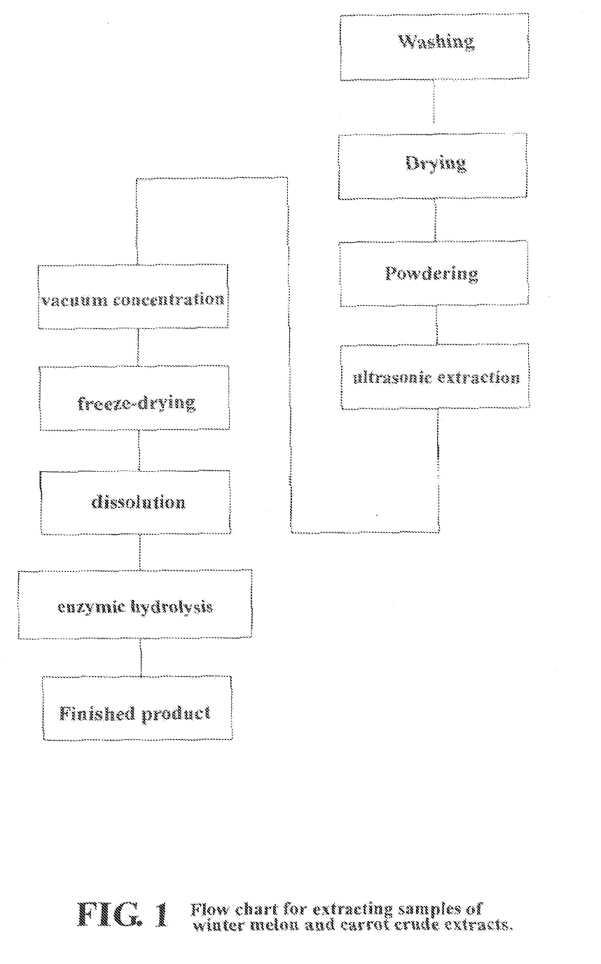

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

The production processes for ACEI product in accordance with the present invention comprising: washing, drying, powdering, ultrasonic extraction, vacuum concentration, freeze-drying, dissolution as well as enzymic hydrolysis etc, and with reference to Figure the procedures and contents are as follows:

A) Washing: Cleaning dirt, insects etc, with clean water on the vegetables which are going to be extracted, and in the case of winter melon, washing thoroughly the melon meat and seeds as well;

B) Drying: but it into an oven, with a convent temperature of 50° C., and drying the sample about 48 hours;

C) Powdering: Grinding dried vegetables to 200 mesh;

D) Ultrasonic extraction: Taking water and alcohol as dissolvent, under a frequency ranging: 28˜160 KHz, and the setting value is at 40 KHz;

E) Vacuum concentration: Performing a vacuum concentration process with a constant temperature of 50° C.;

F) Freeze-Drying: Under the condition of −45° C., 5*10⁻³ mmHg;

G) Dissolution: Performing a hydrolysis process with the combination of protease, pepsin, trypsin, and then diluted by water with a 10 times volume, and having such sample with concentration of about 0.03 mg/mL;

H) Hydrolysis enzyme: Taking the said enzyme combination and adding 0.1% (v/v), and proceeding a hydrolysis process at a temperature of 37° C. for about 2 hours;

I) finished product: It is applicable to acquire the vegetable extract liquid with high-content of ACEI, so as to be provided as an additive for the functional foods.

The present invention has referred to and modified the measuring methods proposed by scholars Cushman and Cheung (1971) as well as Wu and Ding (2002), wherein the measurement principles is that the substrate of hippury-L-histidy-L-Leucine (HHL) would be decomposed into Hippuric acid (HA) and Histidy-Leucine (HL) by ACE, and supposed the vegetable crude extract having ACE effect, then the content in Hippuric acid (HA) and Histidy-Leucine (HL) will be reduced a lot where Hippuric acid (HA) distilled by ACE will absorb ultraviolet rays dramatically, therefore, it is applicable to measure the generation amount per unit time before/after adding Hippuric acid (HA) by the utilization of HPLC, and in this way, the inhibition capability for ACEI will be revealable accordingly. Then preparing 31 μl vegetables crude extract liquid, adding 50 μl ACE solution, and predicating 10 minutes under 37° C., and then adding 0.3 ml HHL solution, oscillating in water bath at 37° C. for 30 minutes, afterwards, adding 5N HCl to terminate the relevant enzyme reaction, and again extracting Hippuric acid product with ethyl acetate, and taking 1 ml upper-level liquid after proceeding a 7500×g centrifugal process, and heating in boiling water bath for about one hour, in order to eliminate ethyl acetate, and finally, dissolving it with 1 mL distilled water, and evaluating ACE inhibition capability with HPLC. During the experiment, the blank group has been added 5N HCl advance to terminate enzyme reaction before adding any enzyme, and instead while the control group replaces vegetable crude extract liquid with the borate buffer.

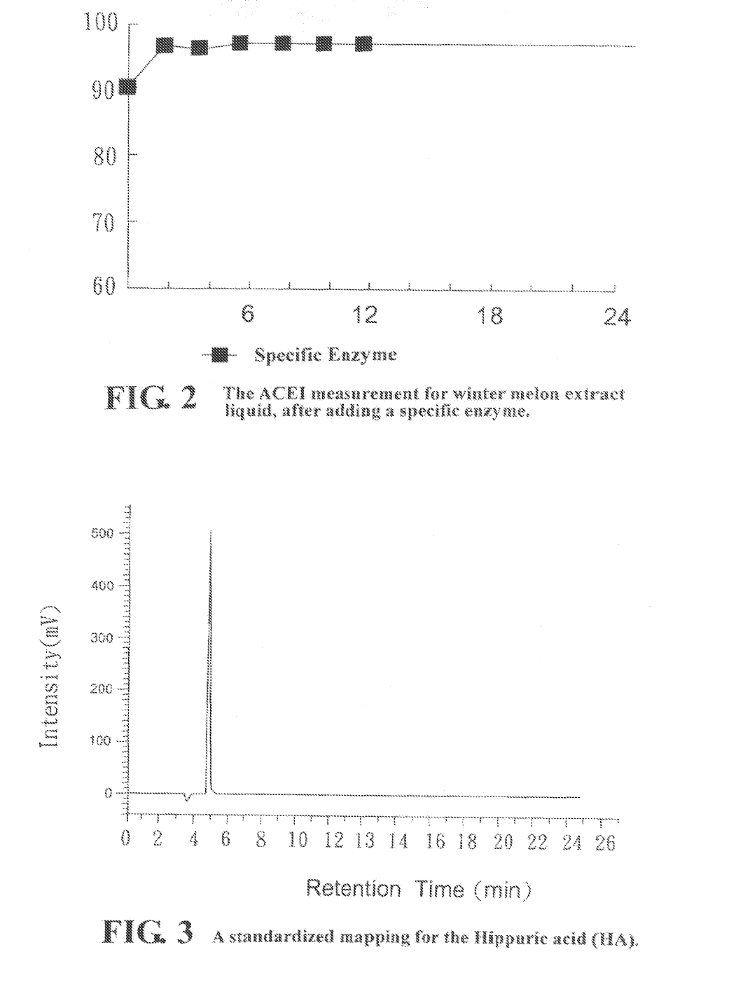

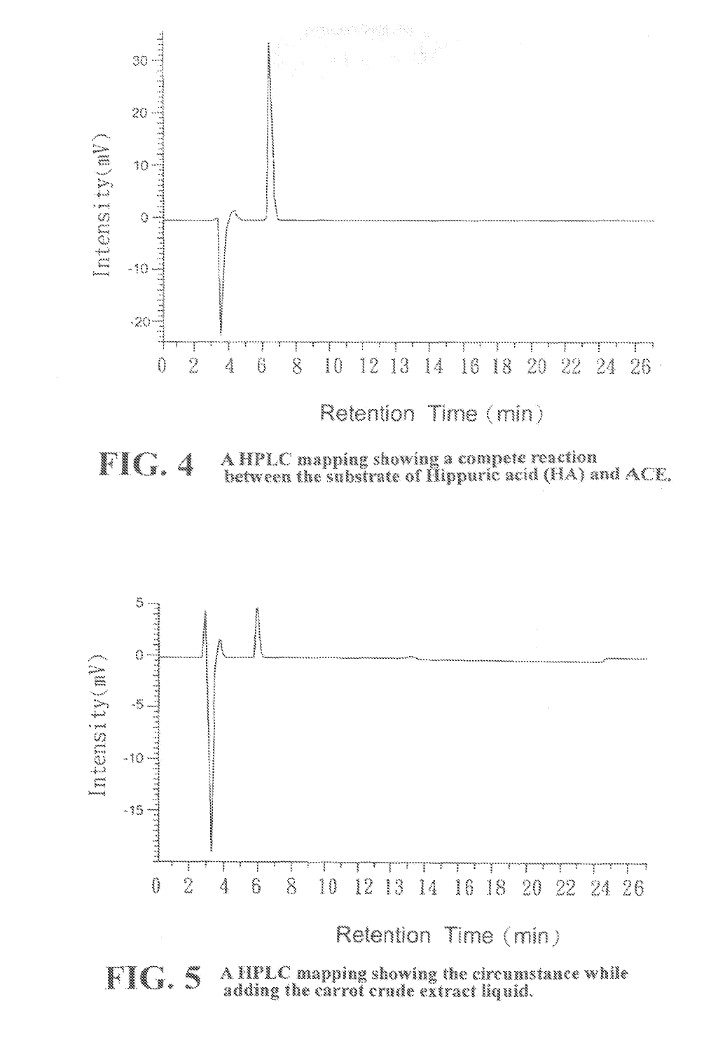

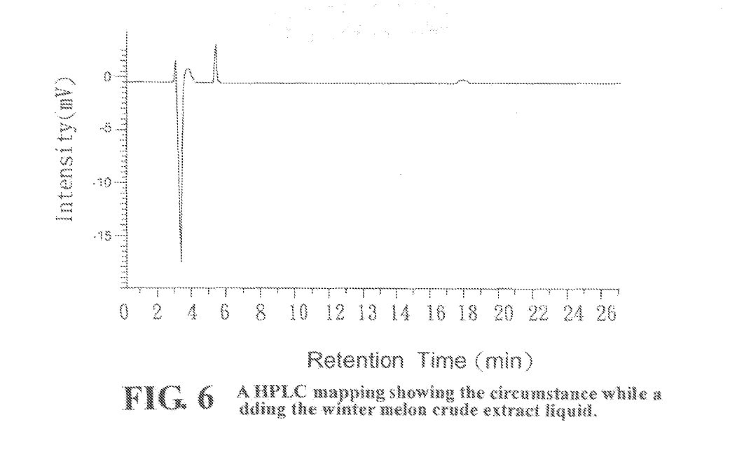

The winter melon crude extract liquid with high ACEI is obtained by ultrasonic method, which is prepared in specific ratio with various enzyme hydrolysate, and adding enzyme with a ratio of 1:100, and then performing the hydrolysis process in different timing with enzyme in the most suitable pH value, and the results are shown in FIG. 2, where it is found that the ACEI capability is enhancing 10%, and content of ACEI is nearly 98%, and while referring to the following FIGS. 3, 4, 5, and 6, wherein FIG. 3 is a standardized mapping for the Hippuric acid (HA), FIG. 4 is a HPLC mapping showing a complete reaction between the substrate of Hippuric acid (HA) and ACE, FIG. 5 and FIG. 6 am the HPLC mapping showing the circumstance while adding the vegetable crude extinct liquids; So as to evaluate ACEI capability among various vegetable crude extract liquids, in which, most of vegetable crude extract liquids are processed with water (0.3 mg/mL), the ACEI capability is about 90%, while it is comparing with Yang (2003), then it is applicable to predict the measurement in concentration of semi-inhibition with the same vegetable crude extract liquid, because it is added with specific enzyme in addition to the ultrasonic extraction technique in accordance with the present invention, wherein the protein molecule has been degraded partially, therefore it is available to perform, in concentration of semi-inhibition, and hence acquiring a higher content, of ACEI while being re-distilled again with specific enzyme hydrolysate, despite the previous research reports had also revealed that ultrasonic process is capable to degrade matter with a large molecule structure.

The said ultrasonic extract together with winter melon hydrolysate produced with a specific enzyme, so that it is applicable to carry out the Peptide Sequence Analysis by means of the EDMAN degradation (Wu and Ding, 2002; Kuba et al., 2003), wherein the N-end of protein widen is similar to dansylation is modified with PTTC (phenylisothiocyanate) so as to generate PTH derivate and, in addition, the N-end amino acid is cut off after performing a Edman Reaction, and then determining the category of amino acid by the HPLC Chromatography, where the remaining protein segment is able to be performed the secondary Edman Reaction, and whereat the sequence after sequencing is Glu-Lys-Thr-Lys-Leu-Gly-Ala.

Additionally upon performing the living organism experiments, we have bought the 6-week old rats suffered man diastolic hypertension disease SHR) (Yoshii et al. 2001), weighted in 130˜150 gram, in a total quantity of 16, where they were firstly temporarily classified, by the weight, into cages, and each cage contains 2˜3 rats, while training and quarantining them for about one week, and then picking up 14 rats in health condition for relevant experiments; while during this period, each cage contains one rat only, and the cage that we used at this stage has been changed to the meshed type stainless steel cage, under automatic controlled temperature of 20˜25° C., in relative humidity 50˜70% for controlling 12 hours under bright/dark condition switching in turn, while during the annual experiment period of 4 weeks, the weight and feeding amount for each animal were recorded in audition to the their growth condition.

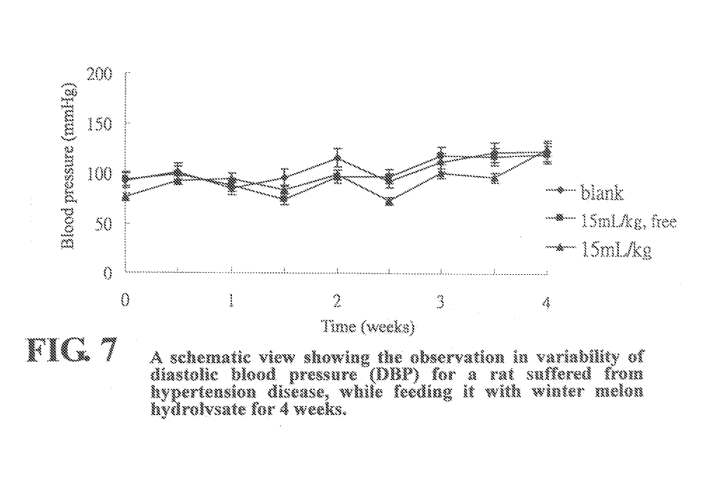

As shown in FIG. 7, after feeding for a period of 28 days, and measuring blood pressure of rat twice in a week, and it is found rival the rat suffered from hypertension disease, after having winter melon crude extract liquid, has a obvious reduction in diastolic blood pressure (DBP) about 20 mmHg, meanwhile the ACE activity inside its organs is lower than that of control group.

Although the present invention has been described with reference to the preferred embodiment thereof, it is apparent to those skilled in the art that a variety of modifications and changes may be made without departing from the scope of the present invention which is intended to be defined by the appended claims.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by mow skilled in the art without departing in any way from the spirit of the present invention.

I claim:

1. A kind of method for producing functional food aimed at blood pressure reduction from the hydrolyzed vegetable solution, related to production, methods for producing high-performance angiotensin converting enzyme inhibitor (ACEI) from vegetables by the following processes, such as washing, drying, powdering, ultrasonic extraction, vacuum concentration, freeze-drying, dissolution as well as enzymic hydrolysis etc. and, in addition, the said ACEI is acting as a kind of food additive.

2. A kind of method for producing functional food aimed at blood pressure reduction from the hydrolyzed vegetable solution as claimed in claim 1, wherein the drying condition is to dry it at a temperature of 50° C. for 48 hours.

3. A kind of method for producing functional food aimed at blood pressure reduction from the hydrolyzed vegetable solution as claimed in claim 1, wherein the powdering condition is up to 200 mesh.

4. A kind of method for producing functional food aimed, at blood pressure reduction from the hydrolyzed vegetable solution as claimed in claim 1, wherein the ultrasonic extraction condition is utilizing a combination of water and alcohol as a dissolvent, and the ultrasonic oscillation frequency ranges between 28 KHz˜160 KHz, and washing power is about 200 W.

5. A kind of method for producing functional food aimed at blood pressure reduction from the hydrolyzed vegetable solution as claimed in claim 1, wherein the vacuum concentration is performed with a heating temperature of 50° C.

6. A kind of method for producing functional food aimed at blood pressure reduction from the hydrolyzed vegetable solution as claimed in claim 1, wherein the freeze-drying condition is at a temperature of 45° C. under 50*10⁻³ mmHg.

7. A kind of method for producing functional food aimed at blood pressure reduction trout the hydrolyzed vegetable solution as claimed in claim 1, wherein the dissolution condition is to perform a hydrolysis process with the combination of protease, pepsin, trypsin, and then diluted by water with a 10 times volume, and while under this condition, such sample's concentration is about 0.03 mg/mL.

8. A kind of method for producing functional food aimed at blood pressure reduction from the hydrolyzed vegetable solution as claimed in claim 1, wherein it is added with 0.1% (v/v) of the combination of protease, pepsin, trypsin, and then performed the hydrolysis process at a temperature of 37° C. for about 2 hours.

9. A kind of method for producing functional food aimed at blood pressure reduction from the vegetable hydrolysate as claimed in claim 1, wherein the winter melon hydrolysate is produced after performing the ultrasonic extraction process with a specific enzyme, so that it is applicable to carry out the Peptide Sequence Analysis by means of the Edman degradation (Wu and Ding, 2002; Kuba et al., 2003), whereat the sequence after sequencing is Glu-Lys-Thr-Lys-Leu-Gly-Ala.
